(19) United States
(12) Patent Application Publication
(10) Pub. No.: US 2013/0245356 A1
(43) Pub. Date: Sep. 19, 2013

(54) HAND HELD SURGICAL DEVICE FOR MANIPULATING AN INTERNAL MAGNET ASSEMBLY WITHIN A PATIENT

(71) Applicants: Raul Fernandez, Arlington, TX (US); Sean P. Conlon, Loveland, OH (US); Richard Bergs, Grand Prairie, TX (US)

(72) Inventors: Raul Fernandez, Arlington, TX (US); Sean P. Conlon, Loveland, OH (US); Richard Bergs, Grand Prairie, TX (US)

(73) Assignees: Board of Regents of The University of Texas System, Austin, TX (US); Ethicon Endo-Surgery, Inc., Cincinnati, OH (US)

(21) Appl. No.: 13/420,818

(22) Filed: Mar. 15, 2012

(51) Int. Cl. A61N 2/12; A61N 2/10

(52) U.S. Cl. 600/12; 600/9

(57) ABSTRACT

A device for manipulating a magnetic coupling force across tissue in response to a monitored coupling force is described. The device includes a magnetic field source assembly that includes at least one fixed magnet and a rotatable magnet positioned within a cavity defined by the fixed magnet that provide an external magnetic field source for providing a magnetic field across tissue. An actuation assembly is operatively connected to the magnetic field force assembly. A sensor is provided that senses a magnetic coupling force and communicates changes therein to a controller which directs the accuation assembly to adjust the speed of rotation of the rotatable magnet in response to the sensed changes in magnetic coupling force to effect a change of magnetic flux generated by the rotatable magnet.

### BACKGROUND

i. Field of the Invention

The present application relates to methods and devices for minimally invasive therapeutic, diagnostic, or surgical procedures and, more particularly, to magnetic guidance systems for use in minimally invasive procedures.

ii. Description of the Related Art

In a minimally invasive therapeutic, diagnostic, and surgical procedures, such as laparoscopic surgery, a surgeon may place one or more small ports into a patient's abdomen to gain access into the abdominal cavity of the patient. A surgeon may use, for example, a port for insufflating the abdominal cavity to create space, a port for introducing a laparoscope for viewing, and a number of other ports for introducing surgical instruments for operating on tissue. Other minimally invasive procedures include natural orifice transluminal endoscopic surgery (NOTES) wherein surgical instruments and viewing devices are introduced into a patient's body through, for example, the mouth, nose, or rectum. The benefits of minimally invasive procedures compared to open surgery procedures for treating certain types of wounds and diseases are now well-known to include faster recovery time and less pain for the patient, better outcomes, and lower overall costs.

Magnetic anchoring and guidance systems (MAGS) have been developed for use in minimally invasive procedures. MAGS include an internal device attached in some manner to a surgical instrument, endoscope, laparoscope or other camera or viewing device, and an external hand held device for controlling the movement of the internal device. Each of the external and internal devices has magnets which are magnetically coupled to each other across, for example, a patient's abdominal wall. In the current systems, the external magnet may be adjusted by varying the height of the external magnet.

The foregoing discussion is intended only to illustrate various aspects of the related art in the field of the invention at the time, and should not be taken as a disavowal of claim scope.

### SUMMARY

A device is described herein for manipulating a magnetic coupling force across tissue based on the monitored coupling force generated between externally and internally disposed magnets. In one embodiment, the device includes a magnetic field source assembly that comprises a first magnetic field source for providing a magnetic field across tissue. The first magnetic field provides a magnetic coupling force between the first magnetic field source and an object that provides or is associated with a second magnetic field. The device also includes an actuation assembly operatively connected to the magnetic field force assembly for adjusting the movement of the first magnetic field source, and a magnetic coupling force monitor.

In certain embodiments, the device for manipulating a magnetic coupling force across tissue comprises a magnetic field source assembly comprising a first magnetic field source positioned in use on one side of tissue and for providing, in use, a magnetic field across the tissue. The first magnetic field source provides a magnetic coupling force between the first magnetic field source and an object positioned, in use, on the opposing side of the tissue which provides, in use, a second magnetic field source. The first magnetic field source comprises at least one fixed magnet and at least one rotatable magnet. The device also includes an actuation assembly operatively connected to the magnetic field force assembly for rotating the rotatable magnet to adjust magnetic flux generated by the first magnetic field source. The device further includes a magnetic force monitoring system for sensing changes in the magnetic coupling force. The monitoring system is in operative communication with the actuation assembly for controlling the actuation thereof in response to the changes in the magnetic coupling force.

In various embodiments, the magnetic field source assembly may further include a magnet suspension member, and the fixed magnet may be operatively suspended from the suspension member. The fixed magnet may define a cavity therein for receiving the rotatable magnet. The actuation assembly may include a driver for effecting rotation of the rotatable magnet, a rack and pinion gear set for driving the driver, and an actuator to actuate the rack and pinion gear set.

The actuator may actuate the rack and pinion gear set, for example, in response to signals from the magnetic force monitoring system. In various embodiments, the actuator may be a motor having a reciprocating arm operatively connected to the rack of the rack and pinion gear set such that reciprocation of the arm effects reciprocal linear motion of the rack. In various embodiments, the pinion gear may be operatively connected to the rack such that the linear motion of the rack is translated into rotational movement of the pinion gear, and the driver may be a drive shaft operatively connected to the pinion gear such that rotation of the pinion gear effects rotation of the drive shaft. The motion of the reciprocating arm may be in stepped increments or may be continuous.

The magnetic coupling force monitor may comprise a sensor plate, a sensor positioned adjacent the sensor plate for measuring changes in the magnetic coupling force between the first magnetic field source and the second magnetic field source and for transmitting signals representative of the measured change in the magnetic coupling force, a control unit for receiving the signals from the sensor, and a processor in communication with the control unit for converting the received signals to output signals for signaling the actuator to adjust the direction of rotation of the rotatable magnet until a predetermined magnetic coupling force is measured by the sensor.

The device may also include in certain embodiments, a suspension member attached to the at least one fixed magnet, and a support member positioned proximally to the suspension member for housing the rack and pinion gear set and a proximal portion of the driver. The support member may have a surface for supporting the sensor. The sensor plate may be positioned proximally to the support member in a facing relationship to the sensor. In various embodiments, at least a portion of the sensor plate is in contact with the sensor.

A plurality of elevation members may be provided. Each elevation member may be slidingly connected at a proximal end thereof to the sensor plate and at a distal end thereof to the suspension member. Each elevation member may have a smooth proximal portion for sliding engagement with the support member and the sensor plate for allowing the sensor plate to move between a rest position and positions of applied force relative to the sensor. In various embodiments, an increased magnetic coupling force operatively exerts a distally directed force on the sensor plate moving the sensor plate from the rest position to an applied force position relative to the sensor, wherein the change in the force exerted on the sensor is communicated to the actuator.

The sensor and the actuator may be in communication with a control unit for matching the sensed change in force exerted on the sensor to a predetermined desirable force within a range of acceptable forces. In such embodiments, the control unit communicates commands to the actuator to adjust the rotation of the rotatable magnet, which adjusts the magnetic flux generated by the first magnetic field source if the sensed force exerted on the sensor does not match the predetermined desirable force.

In certain aspects, the device for manipulating a magnetic coupling force across tissue may comprise a suspension block and a magnetic field source assembly comprising at least one magnet fixedly suspended from the suspension block and at least one rotatable magnet positioned within a cavity defined within the fixed magnet. In this aspect, the device further includes a support block, an actuation assembly and a magnetic force monitoring system. The actuation assembly comprises a driver for effecting rotation of the rotatable magnet to adjust magnetic flux generated by the magnetic field source assembly, a rack and pinion gear set housed in the support block for driving the driver, and an actuator for actuating the rack and pinion gear set. The magnetic force monitoring system comprises a sensor supported by the support block and a sensor plate. The sensor plate may be positioned proximally in a facing relationship relative to the sensor such that at least a portion of the sensor plate is in contact with the sensor. In this aspect, the device includes a plurality of elevation members, each of which is slidingly connected at a proximal end thereof to the sensor plate and at a distal end thereof to the suspension member. Each elevation member in this embodiment has a smooth proximal portion for sliding engagement with the support member and the sensor plate for allowing the sensor plate to move between a rest position and positions of applied force relative to the sensor. The sensor may be calibrated to sense any change in the force exerted on the sensor by the sensor plate. A communication circuit from the sensor to the actuator controls the actuation of the actuator in response to the monitored changes in force.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1A is a perspective view of an embodiment of a hand held surgical manipulation device and FIG. 1B shows the manipulation device of FIG. 1A positioned on the exterior of a patient's torso magnetically positioning a surgical tool placed inside the patient opposite the external manipulation device.

FIG. 2 is a rear view of an embodiment of the device of FIG. 1 with the housing and top cover removed.

FIG. 3. is a perspective view of the bottom of an embodiment of the device of FIG. 2.

FIG. 4 is a front section view through an embodiment of the device of FIG. 1.

FIG. 5 is a side section view through an embodiment of the device of FIG. 1.

FIG. 6 is a front perspective section view through an embodiment of the device of FIG. 2 with the top cover removed.

FIG. 7 is a rear perspective view of an embodiment of the device of FIG. 1 showing a transparent support block with the top cover removed.

FIG. 8 is a perspective view of the device of FIG. 1 with the top cover and support block removed.

FIG. 9 is a schematic view of certain components of an embodiment of a sensor system usable in the hand held manipulation device.

FIG. 10 is a graph showing the change in the coupling force (labeled attraction force) with the change in vertical face distance between the internal and external magnetic field sources.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

In describing and claiming the present invention, the following terminology will be used in accordance with the definitions set out below.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument or component described that is closer to the clinician and the term “distal” refers to the portion located farther from the clinician. It will be further appreciated that for conciseness and clarity, spatial terms such as “vertical,” “horizontal,” “up,” and “down”, “upper” and “lower”, “top” and “bottom”, and the like, may be used herein with respect to the illustrated embodiments. However, surgical instruments may be used in many orientations and positions, and these terms are not intended to be limiting and absolute.

As used herein, the term “elevational position” with respect to one or more components means the distance of such component or components above a floor or ground or bottom position of another component or reference point without regard to the spatial orientation of the respective components.

As used herein, the term “biocompatible” includes any material that is compatible with the living tissues and system(s) of a patient by not being substantially toxic or injurious and not known to cause immunological rejection. “Biocompatibility” includes the tendency of a material to be biocompatible.

As used herein, the term “operatively connected” with respect to two or more components, means that operation of, movement of, or some action of one component brings about, directly or indirectly, an operation, movement or reaction in the other component or components. Components that are operatively connected may be directly connected, may be indirectly connected to each other with one or more additional components interposed between the two, or may not be connected at all, but within a position such that the operation, movement, or action of one component effects an operation, movement, or reaction in the other component in a causal manner.

As used herein, the term “operatively suspended” with respect to two or more components, means that one component may be directly suspended from another component or may be indirectly suspended from another component with one or more additional components interposed between the two.

As used herein, the term “patient” refers to any human or animal on which a suturing procedure may be performed. As used herein, the term “internal site” of a patient means a lumen, body cavity or other location in a patient's body including, without limitation, sites accessible through natural orifices or through incisions.

The manipulation device 10 is structured to manipulate a magnetic coupling force across living tissue 200 between objects having, or associated with, magnetic fields. The manipulation device 10 may generally include a magnetic field source assembly, a magnetic force monitoring system, and an actuation assembly, including an actuator 18, for adjusting the magnetic coupling force. The magnetic field source assembly generally includes at least one outer fixed magnet 40 and at least one inner, rotatable magnet 48. The magnetic force monitoring system generally includes a sensor plate 68 and a sensor 100 in communication with a controller 160. The actuation assembly may be in the form of a gear assembly that may generally include, in addition to actuator 18, a rack and pinion gear set comprised of rack 110 and pinion gear 88, arms 34 and 22 operatively connecting the rack and pinion gear set to actuator 18 and a drive shaft 44.

Adjustments to the magnetic coupling force may be made in various embodiments of the device 10 by adjustments to the actuator 18 by signals from a control unit 160 in response to the monitored magnetic force. As explained in more detail below, the actuator 18 may adjust the movement of the actuation assembly which results in rotation of the rotatable magnet 48 which adjusts the magnetic field strength.

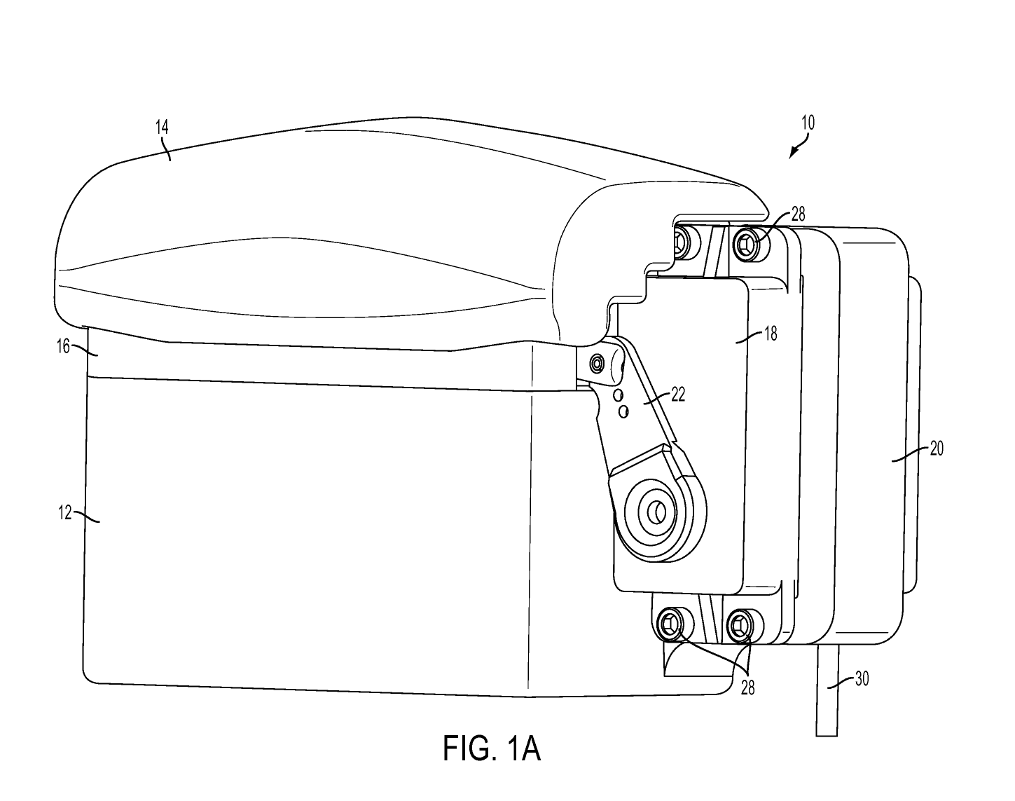

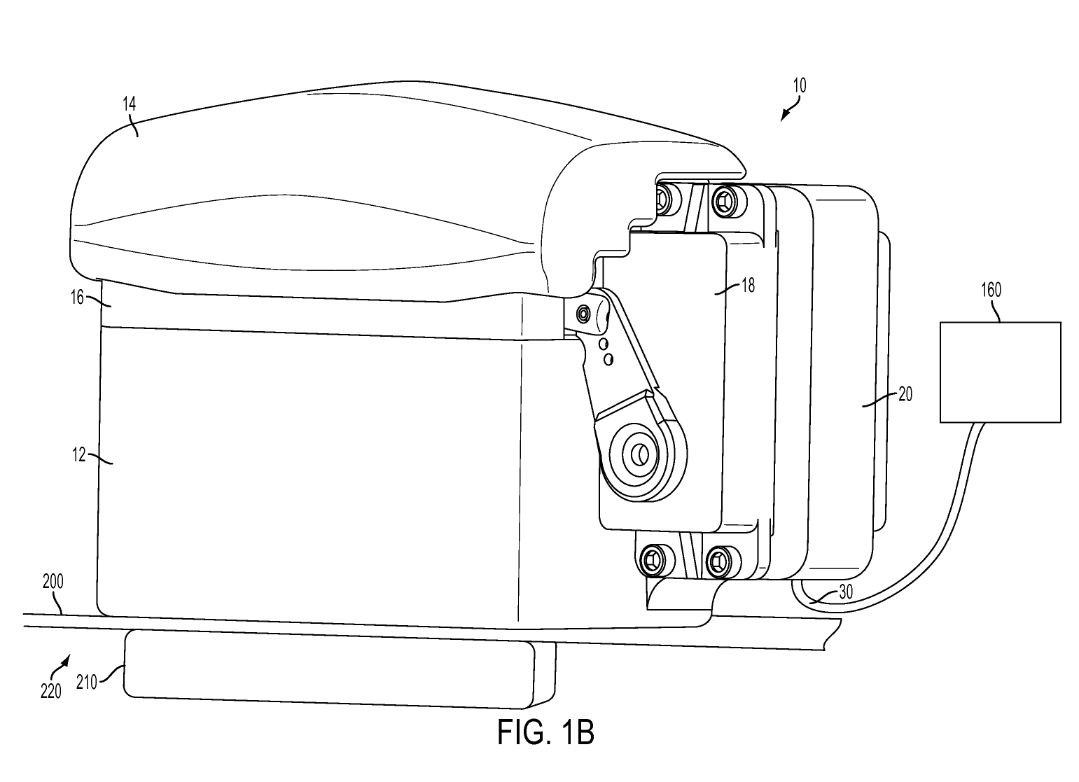

The magnetic field source assembly includes an external magnetic field source that provides a magnetic field across tissue 200. In MAGS applications, there is an object 210, as shown in FIG. 1B, positioned in use on an internal site 220 of a patient, across the tissue 200 (e.g., the abdominal wall or other tissue barrier between the inside and the outside of the patient) from the externally positioned manipulation device 10. The internal object 210 is itself, or is operatively connected to another component that is, a source of an internal magnetic field. The external magnetic field of the magnetic field source assembly and the internal magnetic field source create a magnetic coupling force wherein the internal object 210 is magnetically coupled across the tissue 200 to the magnetic field source of the externally positioned manipulation device 10.

Lateral movement of the manipulation device 10 over the external surface of the tissue 200 causes a similar lateral movement of the internal object 210 on the internal surface of the tissue. If the magnetic coupling force is too strong, however, lateral movement may be difficult due to the resistance to movement by the strongly attracted, magnetically coupled objects, or if too weak the internal object 200 will not remain attached or well controlled by manipulation device 10. Based on the monitored force generated between the external and internal magnetic field sources, the manipulation device 10 described herein enables control of the magnetic coupling force to maintain the force at a level that is strong enough to hold the internal object 210 while allowing lateral movement of the manipulation device 10 and the good control of internal object 210.

Referring to FIGS. 1A and B, an embodiment of a fully assembled manipulation device 10 is shown that includes a housing 12, a support block 16 mounted above housing 12, a side mounted actuator 18 with a control arm 22 extending into support block 16, and a cover 14. In the embodiment shown, actuator 18 may be any suitable actuator, such as a motor, and in particular, a servo motor, DC motor with gear train, a stepper motor, or the like. Actuator 18 may be powered by any suitable DC power supply, a self contained battery, or by a pneumatic or hydraulic power supply. Alternatively, the actuator may itself be a pneumatic or hydraulic motor. Actuator 18 is held to housing 12 by a bracket 20 that extends outwardly from one side of housing 12. Bracket 20 may be an integral part of housing 12 or may be a separate section fastened to housing 12. Actuator 18 may be secured to bracket 20 by any suitable fasteners 28, such as bolts, screws, or clips or may be welded to bracket 20 or directly to housing 12. Actuator 18 may be electrically connected to a controller 160, such as a circuit board via wire 30. Controller 160 may be a separate, distinct unit remotely positioned from manipulation device 10 or may be housed within or mounted to device 10 in the form of an internal circuit board or one or more microchips. Electrical or other communication signals to actuator 18 may be controlled by an external or internal program or algorithm in response to the sensed magnetic coupling force. The program or algorithm controls the movement of arm 22 of actuator 18. Arm 22 may be moved in a continuous manner or in increments as directed by input from controller 160.

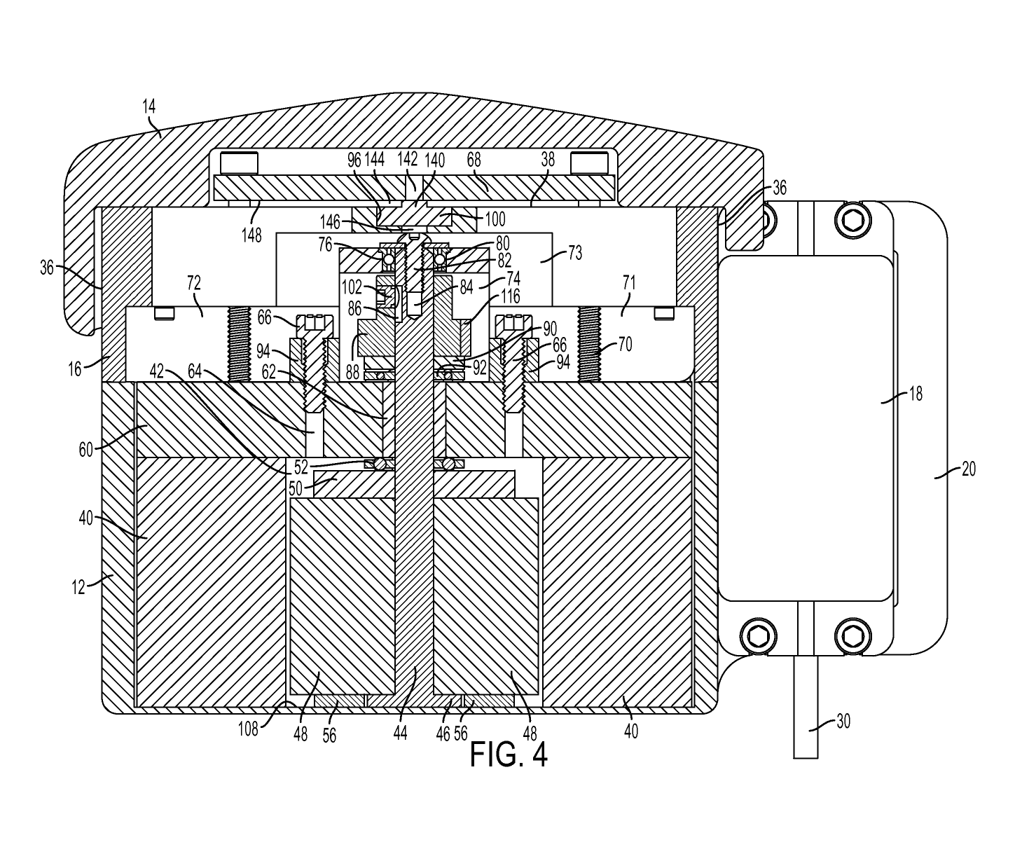

The manipulation device 10 includes a magnetic field source assembly. In various embodiments, the magnetic field source assembly is housed in housing 12 and includes one or more outer magnets 40 and an inner magnet 48. (See for example, FIG. 4) The outer magnet or magnets 40 are suspended from a block 60, for example, by magnetic attraction between the magnets 40 and block 60. In embodiments of the manipulation device 10 having two outer magnets 40, block 60 serves as a bridge to lock the outer magnets 40 into position relative to each other. In certain embodiments, the two outer magnets 40 are of equal and opposite magnetism. When block 60 is made of carbon steel, block 60 acts as a bridge magnetically connecting the North pole on one magnet 40 to the South pole on the opposite magnet 40. Once installed, the magnets 40 and block 60 are magnetically fixed to each other. Those skilled in the art will recognize that other means of attachment between magnets 40 and block 60 may be provided, such as fasteners, in the form of bolts, screws, complementary engagements surfaces and the like.

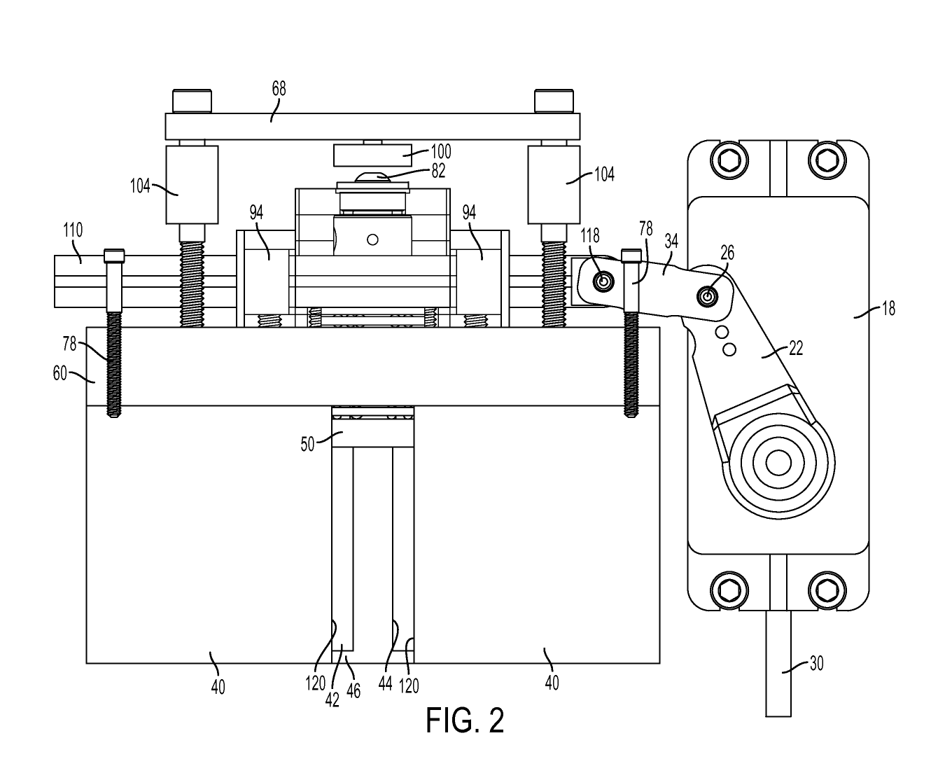

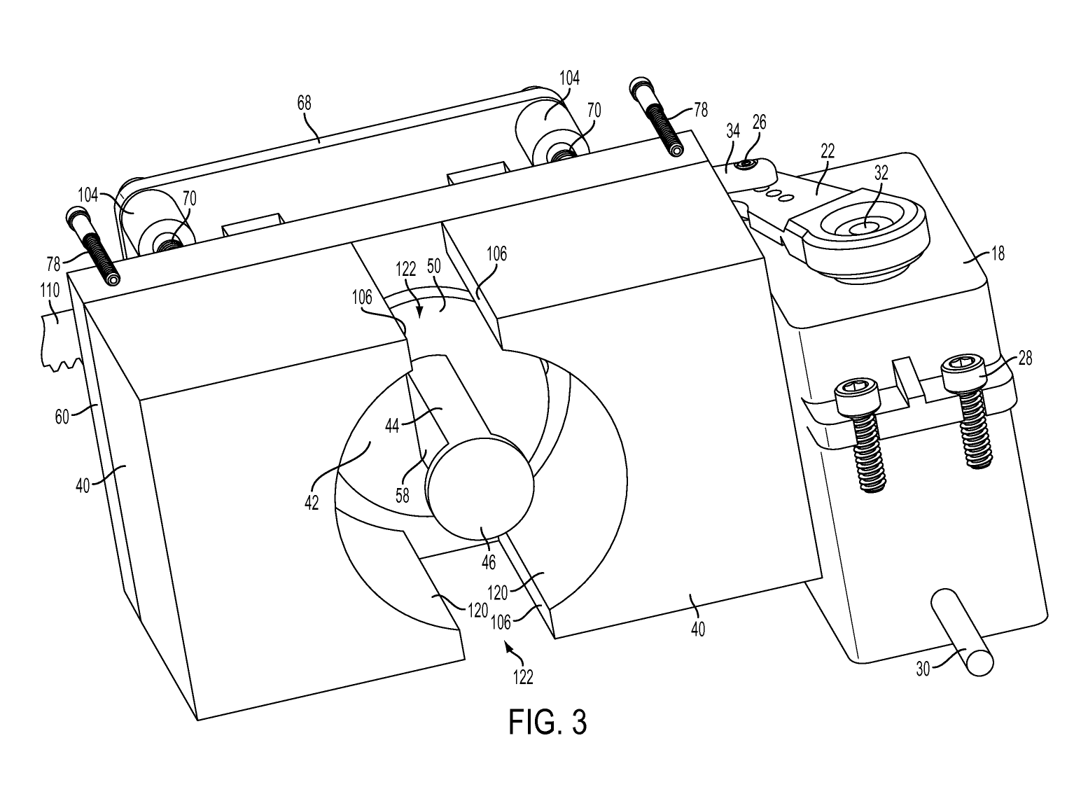

In various embodiments, the outer magnet or magnets 40 define a cavity 42 in which the inner magnet 48 is positioned for movement relative to the outer magnet or magnets 40. Outer magnet 40 may be a single unit defining an open ended cavity 42. Alternatively, as shown in FIGS. 2 and 3, there may be two outer magnets 40 positioned side by side in a facing spaced relationship relative to each other. In certain embodiments, the facing sides 120 of each of the two outer magnets 40 may be concave or arced in configuration, together defining a generally cylindrical cavity 42 with a gap 122 between each of the two opposing ends 106 of each outer magnet 40.

The inner magnet 48 is suspended within the cavity 42 with sufficient space to allow the inner magnet 48 to rotate. In various embodiments, inner magnet 48 rotates within the cavity 42 of the outer magnet or magnets 40. In such embodiments, the rotation of the inner magnet 48 affects the magnetic flux for adjusting the magnetic coupling force between the external magnetic field source assembly and the internal magnetic field source associated with object 210. The configuration of cavity 42 may take any shape that allows inner magnet 48 to freely rotate within the space between the sides of the outer magnet or magnets 40. As shown in the figures, in various embodiments, inner magnet 48 may be cylindrical in shape and is attached to a drive shaft 44 so that inner magnet 48 rotates with drive shaft 44 about a central axis within cavity 42. In various embodiments, the direction and degree of rotation of the inner magnet 48 may be changed from clockwise to counterclockwise and vice versa automatically in response to signals from a sensor 100 to the controller 160 which then, based on the desired coupling force, adjusts the force that the external magnetic field source exerts over the internal magnetic field source and its associated internal object 210 by adjusting the actuation of the gear assembly.

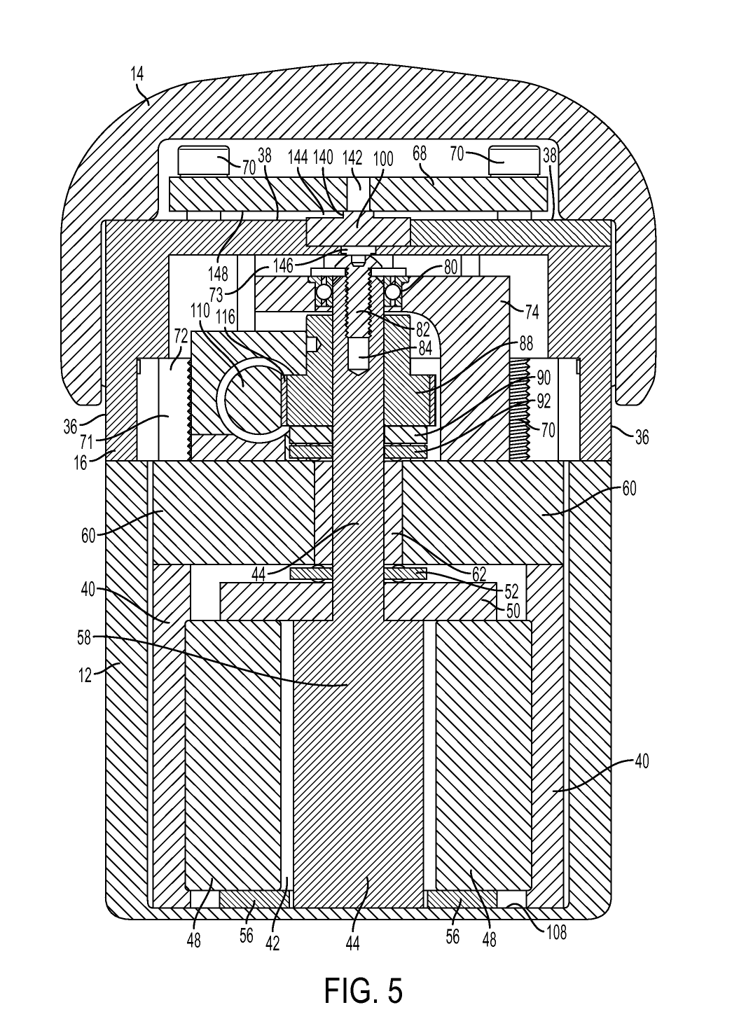

FIGS. 2 and 3 illustrate an exemplary embodiment of the operative connection between the gear assembly and the magnetic field assembly. In various embodiments, the actuation assembly may be in the form of a gear assembly that generally includes drive shaft 44 and a rack and pinion gear set, comprised of rack 110 and pinion gear 88. The magnetic field source assembly, as stated above, includes inner magnet 48, outer magnet or magnets 40, and cavity 42. A distal portion of drive shaft 44 extends into cavity 42 and includes a base section 46 to aid in supporting inner magnet 48 above the floor 108 of housing 12. An annular bushing 56 surrounds base section 46 and sits under inner magnet 48 on the floor 108 of housing 12 within cavity 42. Shaft 44 may be any configuration provided that it can rotate about the axis of rotation within cavity 42. In various embodiments, shaft 44 may have an upper proximal portion that is circular in cross-section and a lower, distal portion 58 that is rectangular in cross-section, as shown in FIGS. 3 and 5, to securely engage inner magnet 48 to drive shaft 44 so that magnet 48 moves with drive shaft 44. In other embodiments, drive shaft 44 may be, for example, generally circular in cross-section along its full length. In such embodiments, inner magnet 48 may be secured to drive shaft 44 or base section 46 or both by one or more pins or other fasteners, or may be press fit onto shaft 44 to ensure that inner magnet 48 moves with drive shaft 44.

An annular bearing surface 50 and rotating annular bearing 52 are shown in the embodiment of FIG. 4 to be positioned within cavity 42 above inner magnet 48 and surrounding drive shaft 44. Bearings 50, 52 above inner magnet 48 and bushing 56 below inner magnet 48 facilitate the ability and ease with which inner magnet 48 rotates within cavity 42.

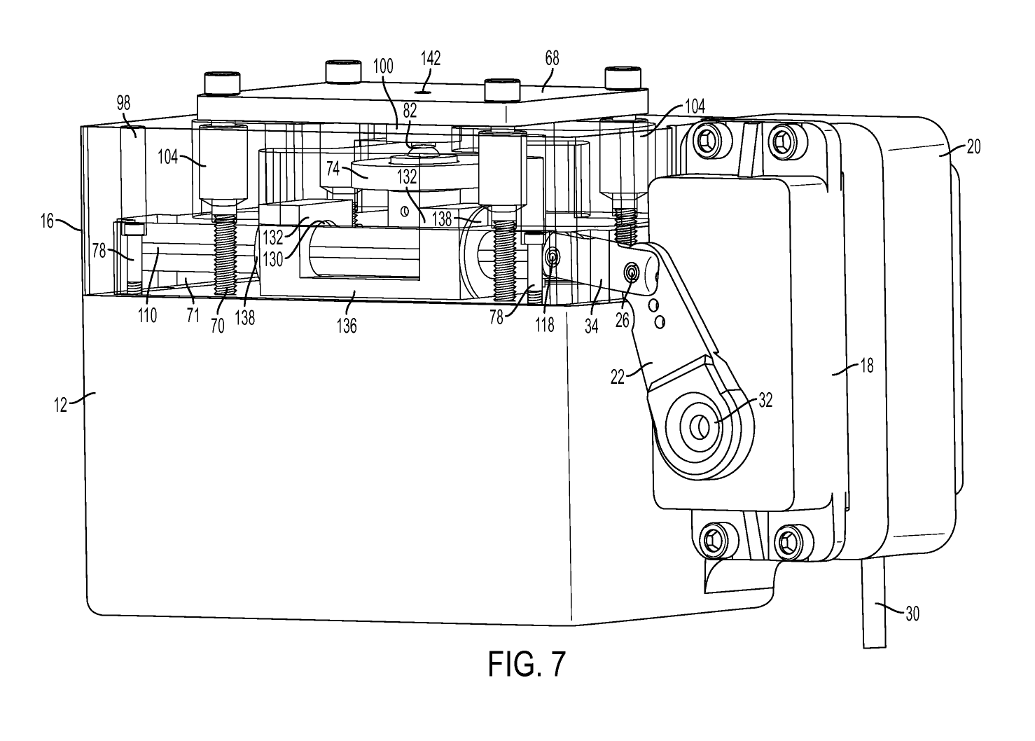

In certain embodiments, as shown in FIGS. 4-6, the additional components of the gear assembly and the magnetic field monitoring system may be housed in and/or supported by support block 16. Block 60 may serve as a platform for support block 16 and various components of the gear assembly. Alternatively, suspension block 60 may serve as a platform for various components of the gear assembly and support block 16 may be attached to housing 12. For example, fasteners 78 may be inserted into bores 98, as shown in FIG. 7, in support block 16 and pass into the upper rim of housing 12. Support block 16 may include side walls 36 and a top surface 38 and define a cavity 72 on its interior. In various embodiments, the cavity 72 may be configured to have differently sized sections 71 and 73 for housing differently sized components of the gear assembly. A well 96 formed in the top surface 38 of support block 16 seats the sensor 100.

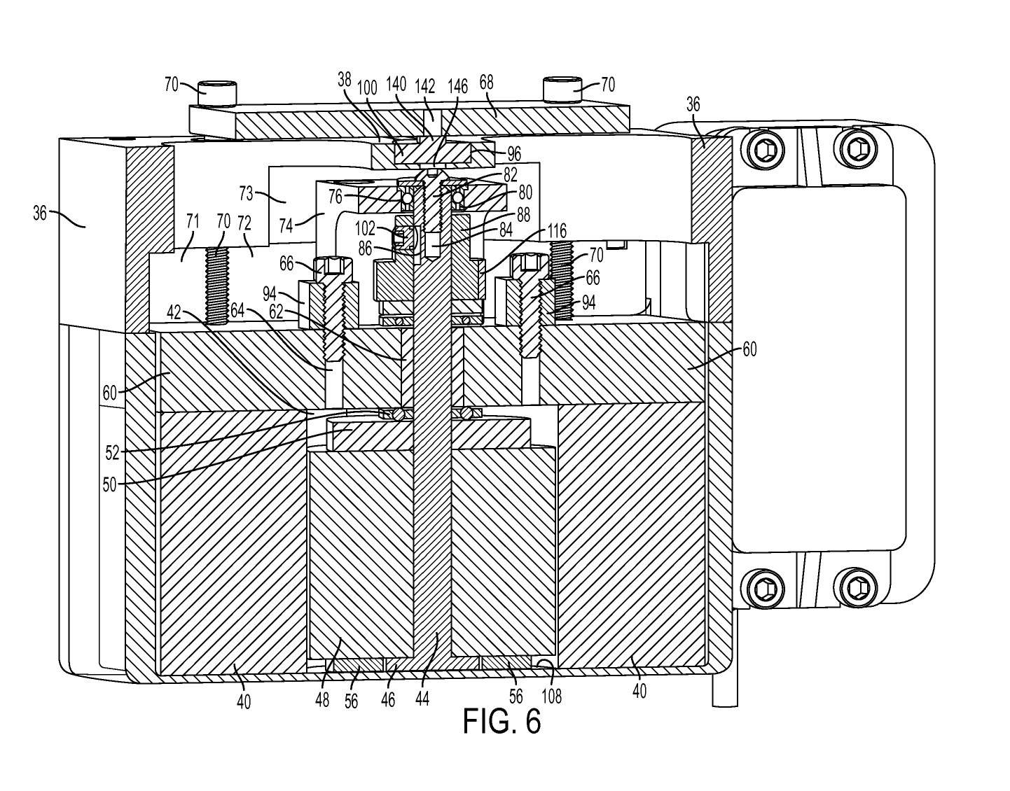

The actuation assembly is operatively connected to and is powered by the actuator 18. In various embodiments, the actuation assembly is a gear assembly that is connected to the actuator 18 through a series of operatively connected interactive gears. Referring to FIGS. 4-6, the gear assembly may include drive shaft 44 and a rack and pinion gear set comprised of pinion gear 88 having gear teeth 116, and rack 110 having gear teeth 114. In the embodiment shown, drive shaft 44 extends from the floor 108 of housing 12 proximally through cavity 42 and through a bushing 62 within an opening, for example, in the form of a bore in suspension block 60, through pinion gear 88 in cavity section 71 of support block 16, and through an opening 76 in the top of a holder, such as L-shaped bracket 74, positioned in cavity section 73 of support block 16. Pinion gear 88 is mounted over drive shaft 44. Pinion gear 88 may be secured to drive shaft 44 by any suitable fastening member, such as set screw 102 which is shown in FIG. 6 extending into a recess 86 along a side near the proximal end of drive shaft 44. A bearing surface, for example, roller ball bearings 80, sits above pinion gear 88 within the opening 76 in L-shaped bracket 74 surrounding drive shaft 44. Additional bearing surfaces 90 and 92 sit under pinion gear 88, also surrounding drive shaft 44. A set screw 82 extending into a central longitudinal bore 84 in the proximal end of drive shaft 44 locks drive shaft 44 and roller bearings 80 to the top of L-shaped bracket 74, pulling this portion of the gear assembly together. A hole 146 in block 16 through the well 96 provides access for a tool to adjust set screw 82 if necessary during assembly.

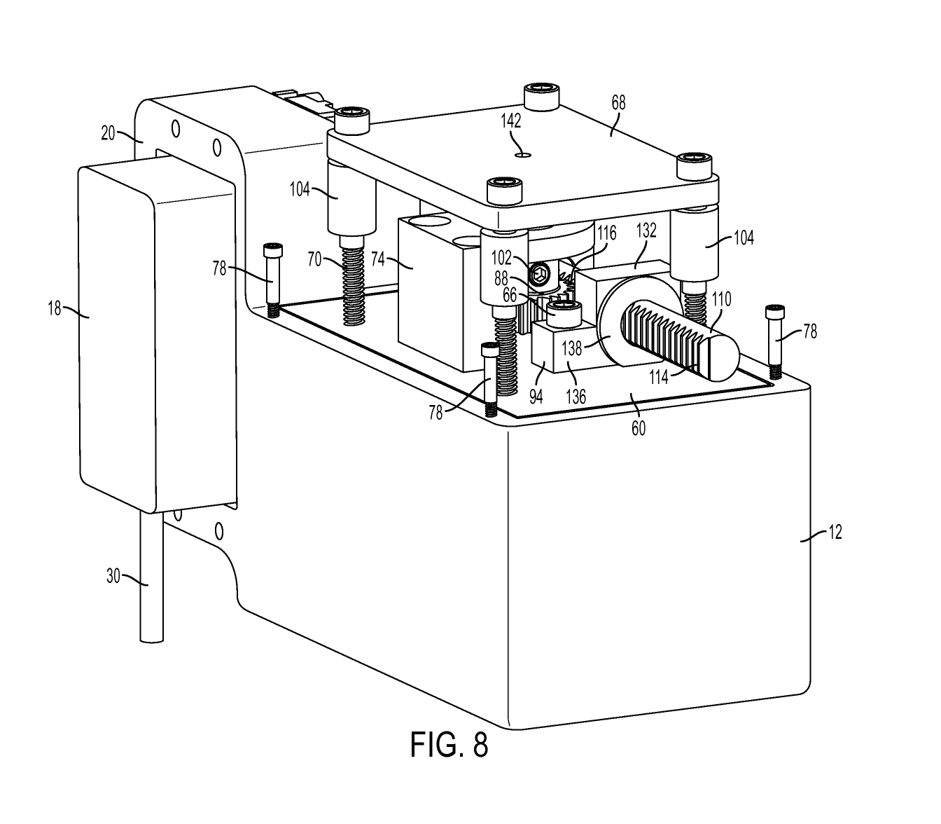

As shown in the embodiment of FIGS. 2, 7, and 8, the gear assembly may include a rack 110 pivotally connected at one end at pivot point 118 to arm 34. Arm 34 is pivotally connected at pivot point 26 to arm 22 and arm 22 is pivotally connected at pivot point 32 to actuator 18. Rack 110 passes through openings 130 in the upwardly extending sections 132 of support bracket 136 in cavity section 71 of support block 16. Support bracket 136 is attached to suspension block 60 by fasteners 66 which extend through bushing portions 94 of bracket 136 into bore 64. Fasteners 66 may be any suitable fastener, such as screws, bolts, clips and the like. Washers 138 or any suitable bearing surface may be positioned at each opening 130 around rack 110. Actuator 18 may power the reciprocal movement of arm 22 back and forth, towards or away from housing 12, effecting the corresponding movement of arm 34 and the corresponding linear movement of rack 110. Gear teeth 114 on rack 110 engage gear teeth 116 on pinion gear 88. The linear movement of rack 110 is translated into, or effects, rotational movement of pinion gear 88 through engagement of the gear teeth 114 and 116. As described previously, pinion gear 88 is mounted on and/or operatively connected to drive shaft 44, such that the clockwise or counterclockwise rotation of pinion gear 88 causes the clockwise or counterclockwise rotation, respectively, of drive shaft 44. As drive shaft 44 rotates, inner magnet 48 rotates with drive shaft 44 within cavity 42. If arm 22 is moving incrementally and/or moving in a reciprocal motion, inner magnet 48 will move incrementally and/or change its direction of rotation as arm 22 changes direction.

The manipulation device 10 exercises automatic control over the magnetic coupling force. A magnetic coupling force monitor is provided in various embodiments of the manipulation device 10. The magnetic coupling force monitoring system may include a sensor 100 and sensor plate 68. Sensor 100 is supported by support block 16. In certain embodiments, sensor 100 may be seated in a well 96 of support block 16. A post 140 extends proximally from sensor 100. Sensor plate 68 rests on post 140 of sensor 100, above the top surface 38 of support block 16, in contact with sensor 100. A hole 142 through sensor plate 68 is provided for insertion of a tool to adjust sensor 100 during assembly or in use thereafter if necessary.

A plurality of bolts 70, such as the four bolts 70 shown in the figures, pass through openings in sensor plate 68. In the embodiments shown in the figures, bolts 70 have a smooth upper or proximal shoulder and surface and a lower threaded end that engages the suspension block 60. The smooth surface portion passes through openings in plate 68 and through bushings 104. Bushings 104 sit in counter bores in block 16. The smooth portion of each bolt 70 is smaller in diameter than the diameter of the bushing 104 into which the bolt 70 is inserted to provide sufficient clearance so that bolts 70 can slide easily relative to bushings 104. Bolts 70 may also be smaller in diameter than the diameter of the openings in sensor plate 68 through which bolts 70 pass to provide sufficient clearance so that bolts 70 can slide easily relative to sensor plate 68.

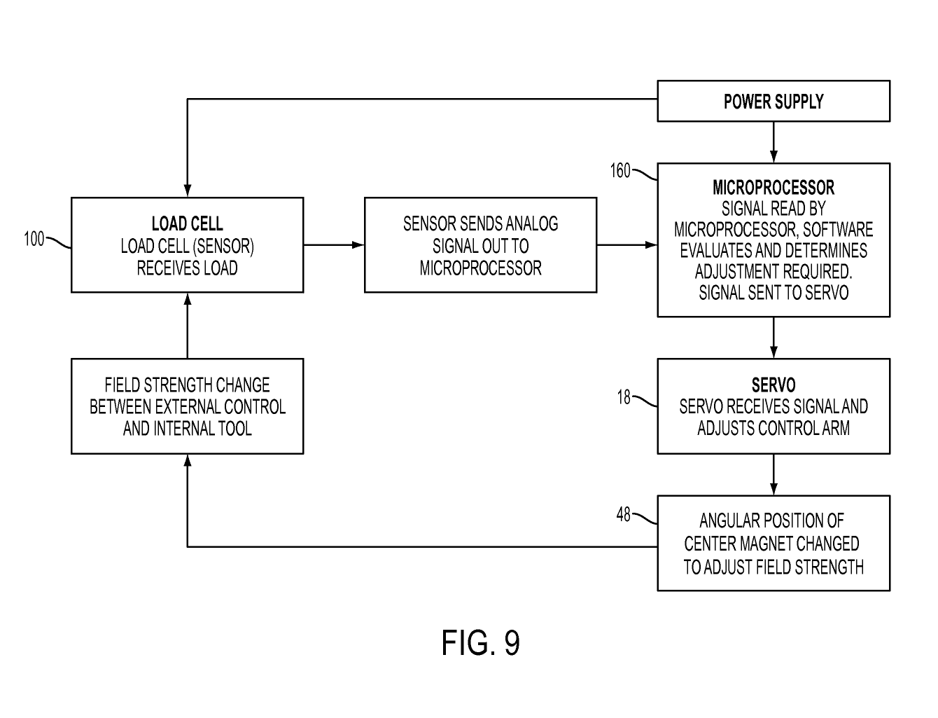

Referring to FIGS. 4-5, in various embodiments, there may be a gap 144 between a portion of the bottom 148 of sensor plate 68 and a portion of the top 38 of support block 16. As described above, sensor plate 68 slides freely relative to bolts 70. Thus, sensor plate 68 is operatively suspended above or “floating” between cover 14 and sensor 100, above but in contact with sensor 100 through post 140. As the magnetic coupling force between the internal magnetic field source and the external magnetic field source assembly increases, the external magnets 40 and 48 are pulled in distally, towards the internal magnetic field source. In various embodiments, magnets 40 are fixedly attached to suspension block 60 by magnetic attraction or other means. The downwardly, or distally directed pull on magnets 40 pulls on blocks 60 and bolts 70, which are connected at their distal ends to block 60. The smooth surface on the upper or proximal portions of bolts 70 allow bolts 70 to slide easily through bushings 104 in support block 16 and the openings in sensor plate 68 with little or no significant resistance, and in certain embodiments, no resistance. As the distally directed force increases, the heads of bolts 70 apply the distally directed force to sensor plate 68 which applies an increased distally directed force to post 140 of sensor 100. As magnets 40 and suspension block 60 are pulled in the distal direction as a result of increased magnetic coupling forces across the tissue 200, sensor plate 68 applies a greater force against sensor 100. Sensor 100 is zeroed out at a value that accounts for the weight of sensor plate 68 and gravity. As the magnetic coupling force between the internal magnetic field source and the external magnetic field source assembly decreases, the magnetic pull from the internal magnetic field source relaxes. The relaxation in force is transferred through magnets 40, blocks 60 and 16 to bolts 70 and sensor plate 68, allowing sensor plate 68 to relax relative to sensor 100. Sensor 100 detects the change in the force applied by sensor plate 68 and communicates the change to controller 160. A wire may extend from sensor 100 to controller 160 to communicate the sensed signal from sensor 100 to controller 160. FIG. 9 illustrates schematically the communication from sensor 100 to controller 160.

As the elevational position of magnets 40 relative to the internal magnetic field source is changed up or down as the magnetic coupling force changes, the force applied to sensor 100 by sensor plate 68 changes accordingly. Because the weight of the sensor plate 68 in a rest position where there is no magnetic coupling force applying a distally directed force on sensor plate 68 is accounted for in calibrating the controller 160, the only force measured when there is a force applied to sensor 100 is the magnetic coupling force between the external magnetic field source and the internal counterpart.

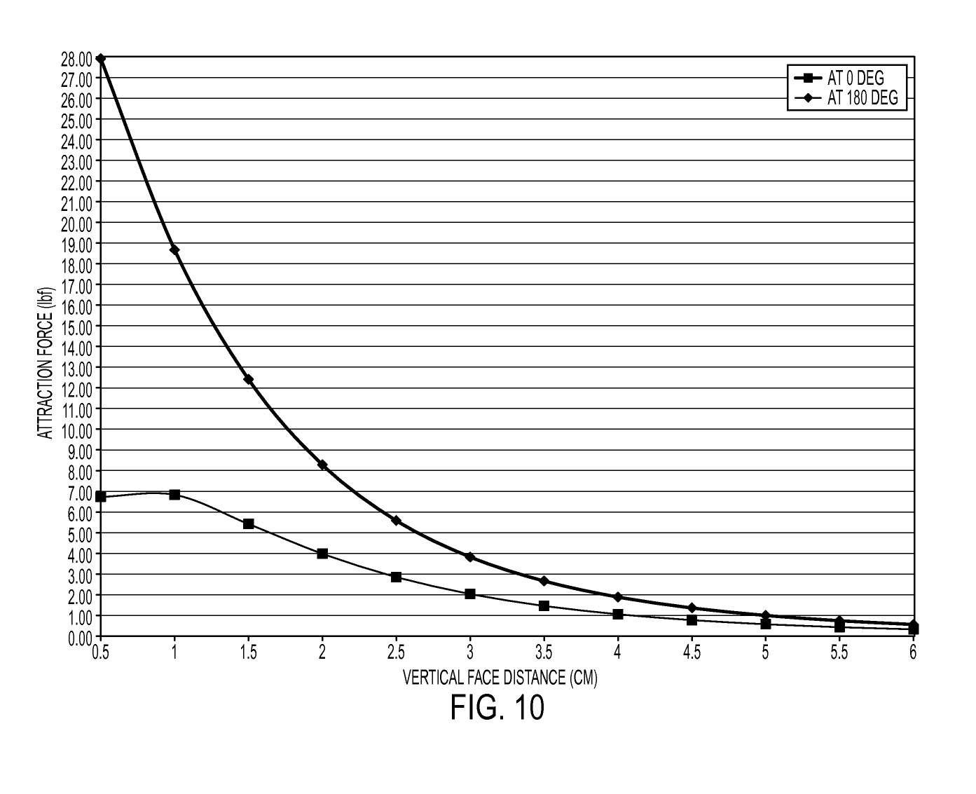

The controller 160 receives a signal from the sensor 100 as to the magnitude of force generated by the magnetic attraction between the external magnetic field source assembly and the internal magnetic field source associated with object 210. As the thickness of tissue 200 gets smaller, the field strength becomes stronger thereby increasing the force on sensor 100. Conversely, as the thickness of tissue 200 gets larger the magnetic field strength becomes weaker reducing the force on sensor 100. For example, at a distance of 5 mm between the vertical faces of the external and internal magnetic field sources, at about 180 degrees of rotation, the load may be 28 lbs, and at zero degrees of rotation, the load may be at 7 lbs. A graph is provided in FIG. 10 showing the change in the coupling force (labeled attraction force) with the change in vertical face distance between the internal and external magnetic field sources. Data is shown for rotatable magnet 48 when at 0 and 180 degrees of rotation. It should be understood, however, that 0 and 180 degrees are arbitrary. Zero is representative of low/off, and 180 is representative of more power. The force output of this embodiment can be anywhere between these two extremes, i.e., 180 is the maximum and zero is the minimum. The result is symmetric, anything less than 180 degrees is equal to that angle over 180 degrees, e.g., the force at 90 and 270 degrees are equal, both in scale and sign. Only the angle matters. The direction of the angle does not matter in changing the magnetic flux generated by the rotatable magnet 48.

The sensor 100 may be, for example, a transducer, a piezoelectric film sensor, or a load cell. The magnetic coupling force pulls the magnets 40, 48. The sensor 100 senses the force and communicates the sensed force to a control unit 160. The control unit 160 may be or may include a circuit board. The circuit board may, for example, utilize a programmable controller (e.g., EPROM) to analyze signals from the sensor 100. Magnetic field lines are established by the magnetic field between the external and internal magnets, pulling the magnets in the magnet housing 12 down, toward the internal magnets associated with the object 210 within the patient. As the downward pull increases, it increases the force applied by the sensor plate 68 to the sensor 100, causing the sensor 100 to measure and register an increased force against it. The sensor 100 signals the calculated force back to the control unit 160 wirelessly or via circuitry. As stated above, the sensor 100 is adjusted to have a zero point accounting for gravity plus the weight of the sensor plate 68.

Those skilled in the art will appreciate that other types of sensors may be used. A LCD screen may be provided to show the force generation between the internal and external magnets.

If sensor 100 is a load cell type of sensor, for example, it feeds the load signal to a signal conditioner. The load cell 100 is acted upon by the attractive forces between the internal and the external magnets. The load cell 100 strains internally and the resulting strain is measured in terms of electrical resistance, using current provided by any suitable power supply. The signal conditioner, which may be contained within the control unit 160, amplifies the signal from the load cell 100 and then a suitable algorithm may be used to calculate the actual force which is then used to drive the actuator 18 at a calculated speed and duration to adjust gear assembly and thereby adjust the rotation of inner magnet 48. Changes to the direction and degree of rotation of magnet 48 adjust the magnetic flux created by the inner magnet 48.

Control unit 160 is equipped with a receiver to receive the signals from sensor 100. Software analyzes the received signals, and sends output signals to instruct the actuator 18. An exemplary commercially available software program suitable for use with the manipulation device 10 is LabVIEW™ system design software sold by National Instruments Corporation. Actuator 18 may be a servo motor or a stepper type motor which, as explained above, will reciprocate arm 22 to move rack 110 and pinion gear 88 and thereby drive the drive shaft 44, which effects rotation of inner magnet 48 in a direction that will match a predetermined force such as the magnetic field strength between the external and internal magnetic field sources. When the predetermined force is sensed by sensor 100, the sensed signals are communicated to the control unit 160 which, as before, instructs the actuator 18 to stop. The continuous monitoring in use of the magnetic coupling force provides an automatic closed loop feedback system to control the magnetic coupling force. The control unit 160 may be on any suitable printed circuit board that receives analog or digital signals and may be packaged within or external to the housing 12 of the manipulation device 10. FIG. 9 shows a schematic of the signal communication from sensor 100 to the control unit 160 to actuator 18.

The predetermined force will be the minimum force that is necessary to attract and accurately control the internal object 210 associated with the internal magnet. The internal magnet must be held with enough magnetic force to prevent it from falling away from the internal body wall. The maximum amount of force would be less than a force that compresses or squeezes the tissue 200 or prevents control over the internal object 210. Those skilled in the art will appreciate that a range of acceptable force may apply and may vary with the patient. The surgeon has to be able to move the manipulation device 10 relatively easily across the patient's body to control the internal magnet associated with internal object 210 without so much drag that movement is difficult.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, nose, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small—keyhole—incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, nose, anus, and/or vagina) or via a trocar through a relatively small—keyhole—incision incisions (usually 0.5-2.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

All materials used that are in contact with a patient are preferably made of biocompatible materials.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK®bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A device for manipulating a magnetic coupling force across tissue comprising:
a magnetic field source assembly comprising a first magnetic field source positioned in use on one side of tissue and for providing, in use, a magnetic field across the tissue, the first magnetic field source providing a magnetic coupling force between the first magnetic field source and an object positioned, in use, on the opposing side of the tissue and providing, in use, a second magnetic field source;
the first magnetic field source comprising at least one fixed magnet and at least one rotatable magnet;
an actuation assembly operatively connected to the magnetic field force assembly for rotating the rotatable magnet to adjust magnetic flux generated by the first magnetic field source; and
a magnetic force monitoring system for sensing changes in the magnetic coupling force, the monitoring system being in operative communication with the actuation assembly for controlling the actuation thereof in response to the changes in the magnetic coupling force.

2. The device recited in claim 1 wherein the magnetic field source assembly further comprises:
a magnet suspension member, and
the fixed magnet being operatively suspended from the suspension member and defining a cavity therein for receiving the rotatable magnet.

3. The device recited in claim 1 wherein the actuation assembly comprises a driver for effecting rotation of the rotatable magnet, a rack and pinion gear set for driving the driver, and an actuator to actuate the rack and pinion gear set.

4. The device recited in claim 3 wherein the actuator actuates the rack and pinion gear set in response to signals from the magnetic force monitoring system.

5. The device recited in claim 3 wherein:
the actuator is a motor having a reciprocating arm operatively connected to the rack of the rack and pinion gear set such that reciprocation of the arm effects reciprocal linear motion of the rack;
the pinion gear is operatively connected to the rack such that the linear motion of the rack is translated into rotational movement of the pinion gear; and,
the driver is a drive shaft operatively connected to the pinion gear such that rotation of the pinion gear effects rotation of the drive shaft.

6. The device recited in claim 5 wherein the motion of the reciprocating arm is in stepped increments.

7. The device recited in claim 5 wherein the motion of the reciprocating arm is continuous.

8. The device recited in claim 5 wherein the motor actuates the movement of the arm, rack and pinion gear set, and drive shaft in response to signals from the magnetic force monitoring system.

9. The device recited in claim 5 wherein the magnetic coupling force monitor comprises:
a sensor plate;
a sensor positioned adjacent the sensor plate for measuring changes in the magnetic coupling force between the first magnetic field source and the second magnetic field source and for transmitting signals representative of the measured change in the magnetic coupling force;
a control unit for receiving the signals from the sensor; and,
a processor in communication with the control unit for converting the received signals to output signals for signaling the actuator to adjust the direction of rotation of the rotatable magnet until a predetermined magnetic coupling force is measured by the sensor.

10. The device recited in claim 9 further comprising:
a suspension member attached to the at least one fixed magnet;
a support member positioned proximally to the suspension member for housing the rack and pinion gear set and a proximal portion of the driver, the support member having a surface for supporting the sensor;
wherein the sensor plate is positioned proximally to the support member in facing relationship to the sensor and wherein at least a portion of the sensor plate is in contact with the sensor;
a plurality of elevation members each slidingly connected at a proximal end thereof to the sensor plate and at a distal end thereof to the suspension member, each elevation member having a smooth proximal portion for sliding engagement with the support member and the sensor plate for allowing the sensor plate to move between a rest position and positions of applied force relative to the sensor.

11. The device recited in claim 3 wherein magnetic field source assembly further comprises:
a housing;
a magnet suspension member positioned within the housing;
the fixed magnet being operatively suspended from the suspension member and defining a cavity therein for receiving the rotatable magnet; and,
the rotatable magnet being operatively connected to the driver.

12. The device recited in claim 11 wherein there are two fixed magnets suspended from the magnet suspension member and positioned in the housing, each fixed magnet having an arced side in an opposed facing relationship relative to the arced side of the other fixed magnet, the opposing arced sides defining a cylindrical cavity for receiving the movable magnet;
the driver extends through the suspension member into the cylindrical cavity; and,
the rotatable magnet is mounted on the driver for movement with the movement of the driver.

13. The device recited in claim 12 further comprising:
the driver having a distal portion and a proximal portion, the distal portion being positioned in the cylindrical cavity; and,
a support member positioned proximally to the suspension member for housing the rack and pinion gear set and the proximal portion of the driver.

14. The device recited in claim 13 wherein the magnetic coupling force monitor comprises a sensor positioned proximally to the magnetic field source assembly, the sensor being calibrated to sense any change in the force exerted on the sensor, and a communication circuit from the sensor to the actuator to control the actuation of the actuator in response to the monitored changes in force.

15. The device recited in claim 14 wherein the magnetic coupling force monitor further comprises:
a sensor plate positioned proximally to the support member in facing relationship to the sensor, at least a portion of the sensor plate being in contact with the sensor, the sensor and sensor plate movable relative to each other between a spaced position and a contact position;
a plurality of elevation members each slidingly connected at a proximal end thereof to the sensor plate and at a distal end thereof to the suspension member, each elevation member having a smooth proximal portion for sliding engagement with the support member and the sensor plate for allowing the sensor plate to move between a rest position and positions of applied force relative to the sensor.

16. The device recited in claim 15 wherein an increased magnetic coupling force operatively exerts a distally directed force on the sensor plate moving the sensor plate from the rest position to an applied force position relative to the sensor, wherein the change in the force exerted on the sensor is communicated to the actuator.

17. The device recited in claim 16 wherein the sensor and the actuator are in communication with a control unit for matching the sensed change in force exerted on the sensor to a predetermined desirable force within a range of acceptable forces;
the control unit communicating commands to the actuator to adjust the rotation of the rotatable magnet to adjust the magnetic flux generated by the first magnetic field source if the sensed force exerted on the sensor does not match the predetermined desirable force.

18. The device recited in claim 17 wherein the actuator is a motor having a reciprocating arm operatively connected to the rack of the rack and pinion gear set such that reciprocation of the arm effects reciprocal linear motion of the rack;
the pinion gear is operatively connected to the rack such that the linear motion of the rack is translated into rotational movement of the pinion gear; and,
the driver is a drive shaft operatively connected to the pinion gear such that rotation of the pinion gear effects rotation of the drive shaft.

19. The device recited in claim 1 further comprising the object, wherein the object is structured for positioning in use on an internal site of a patient and has associated therewith a second magnetic field source for forming with the first magnetic field force the magnetic coupling force across tissue.

20. A device for manipulating a magnetic coupling force across tissue comprising:
a suspension block;
a magnetic field source assembly comprising at least one magnet fixedly suspended from the suspension block, the fixed magnet defining a cavity therein, and at least one rotatable magnet positioned within the cavity of the at least one fixed magnet;
a support block;
an actuation assembly comprising a driver for effecting rotation of the rotatable magnet to adjust magnetic flux generated by the magnetic field source assembly, a rack and pinion gear set housed in the support block for driving the driver, and an actuator for actuating the rack and pinion gear set; and
a magnetic force monitoring system comprising a sensor supported by the support block, and a sensor plate, the sensor plate being positioned proximally in facing relationship to the sensor, at least a portion of the sensor plate being in contact with the sensor;
a plurality of elevation members each slidingly connected at a proximal end thereof to the sensor plate and at a distal end thereof to the suspension member, each elevation member having a smooth proximal portion for sliding engagement with the support member and the sensor plate for allowing the sensor plate to move between a rest position and positions of applied force relative to the sensor, the sensor being calibrated to sense any change in the force exerted on the sensor by the sensor plate, and a communication circuit from the sensor to the actuator to control the actuation of the actuator in response to the monitored changes in force.
